(19) United States
(12) Patent Application Publication
(10) Pub. No.: US 2002/0022762 A1
(43) Pub. Date: Feb. 21, 2002

(54) Devices and methods for warming and cleaning lenses of optical surgical instruments

(76) Inventors: Richard Beane, Hingham, MA (US); Demetrius Litwin, Westboro, MA (US); Steven Ek, Bolton, MA (US); Allison Niemann, Burlingame, CA (US); Melinda Taylor, North Grafton, MA (US)

Correspondence Address: J. PETER FASSE, Fish & Richardson P.C., 225 Franklin Street, Boston, MA, 02110-2804, US

(21) Appl. No.: 09/788,033

(22) Filed: Feb. 16, 2001

Related U.S. Application Data
(60) Provisional application No. 60/183,467, filed on Feb. 18, 2000.

(51) Int. Cl. A61B 1/00

(52) U.S. Cl. 600/101

(57) ABSTRACT

A lens warming and cleaning device for use with an optical surgical instrument is disclosed. The device includes a heat-conducting tube sized and shaped to receive the lens portion of the instrument, a heating element thermally coupled to an exterior of the tube, and a cleaning member disposed within the tube. The cleaning member is disposed such that when the lens portion of the instrument is inserted into the tube, the lens portion contacts the cleaning member.

### CROSS-REFERENCES TO RELATED APPLICATION

This application claims priority from U.S. provisional Application Ser. No. 60/183,467 filed on Feb. 18, 2000, which is incorporated herein by reference in its entirety.

### FIELD OF THE INVENTION

The invention relates to devices used to warm and clean optical surgical instruments, such as laparoscopes or endoscopes.

### BACKGROUND OF THE INVENTION

In minimally invasive surgical procedures, surgical operations are performed using elongated instruments introduced through one or more small incisions. To allow a surgeon to visualize the operating field, an elongated lens and lighting system, such as a laparoscope or an endoscope, is inserted into the operating field through a separate small incision. The optical instrument's lens is typically coupled to a camera head that relays the scope's image to a television monitor. Since the monitor provides the surgeon's only view of the operating field, a clear, well-defined image is essential.

A common problem in minimally invasive surgical procedures is fogging of the lens on the laparoscope or endoscope. When a lens is inserted into a body cavity, e.g., an insufflated abdomen, the lens is at room temperature. The body cavity, however, is saturated with water vapor escaping from internal tissue and organs. Since the water vapor is typically at or near body temperature, microdroplets of water condense on the colder scope lens, obscuring the surgeon's view of the operating field. When the lens fogs, the surgeon must remove the instrument, clean the lens, and then reinsert the instrument into the operating field, where fogging begins again.

To combat fogging, surgeons often warm the optical instrument by partly immersing the instrument in a warm saline bath both before surgery and during each cleaning. Immersing the instrument can be time consuming, however, since the surgeon must wait for the bath to warm the instrument to a temperature warm enough to prevent condensation, e.g., 37-60° C. In addition, the temperature of saline baths can be difficult to control.

In addition to fogging, a surgeon's view of the operating field can be obscured by bodily fluids, such as blood and tissue collecting on the lens of the optical instrument. Like fogging, covering of the lens with blood or tissue requires that the surgeon remove and clean the instrument, which can cool the instrument and accelerate fogging.

### SUMMARY OF THE INVENTION

The invention relates to devices that both warm and clean only the lens and/or distal portion of an optical instrument, e.g., a laparoscope or endoscope, during minimally invasive surgery. Since the devices warm and clean only the lens bearing distal portion of the instrument, they are compact and inexpensive, and they warm the lens more quickly than a saline bath. The devices can be self-contained units, or can be incorporated into a cannula of a trocar-cannula system.

In general, in one aspect, the invention features a lens warming and cleaning device for use with an optical instrument that has a lens portion. The device includes a heat-conducting tube sized and shaped to receive the lens portion of the optical instrument, a heating element thermally coupled to an exterior of the tube, and a cleaning member disposed within the tube. The cleaning member is disposed such that when the lens portion of the optical instrument is inserted into the tube, the lens portion contacts the cleaning member.

Embodiments of this aspect of the invention can include one or more of the following features. The heat conducting tube can be sized and shaped to receive the lens portion of a laparoscope or an endoscope, and the tube can be constructed from aluminum.

The heating element can include a substance that, when triggered, generates an exothermic reaction. For example, the substance can be a mixture of compounds that generates an exothermic reaction triggered by exposure to oxygen. The heating element can generate sufficient heat to warm the lens portion of the optical instrument to between about 45° C. and 60° C.

The heating element can be a flexible pad that surrounds at least a portion of the tube that receives the lens portion of the optical instrument. The pad can include an attachment mechanism, such as an adhesive, that secures the pad around the tube.

The cleaning member can be a sponge disposed within a distal end of the tube. The device can also include a moistening mechanism, such as a squeezable liquid bottle, that moistens the sponge. The liquid bottle can contain a saline solution with an additive such as an anti-fogging additive or a surfactant.

The device can further include a housing that encases the tube and the heating element. The housing can define an opening configured for insertion of the lens portion of the optical instrument.

In another aspect, the invention features a method of warming and cleaning a lens of an optical instrument. The method includes: (a) obtaining the warming and cleaning device described above; (b) withdrawing the optical instrument from an operating field; (c) inserting a lens portion of the optical instrument into the tube such that the lens contacts the cleaning member; and (d) moving the lens while the lens is in contact with the cleaning member to remove fog and debris from a surface of the lens.

Embodiments of this aspect of the invention can include one or more of the following features. The inserting step can include inserting only a distal, lens-bearing portion of the optical instrument into the tube.

The method can further include removing the optical instrument from the tube, reintroducing the optical instrument into the operating field, and then repeating the withdrawing, inserting, and removing steps when the lens portion again requires cleaning.

In another aspect, the invention features a lens warming device that includes a heat-conducting tube sized and shaped to receive a lens bearing portion of an optical surgical instrument, an absorbent member disposed within the tube, and a heating element thermally coupled to the tube.

In another aspect, the invention features a cannula that includes a body that has a distal end and a proximal end, and that defines a bore for passage of an optical instrument therethrough. Disposed within the bore is a cleaning portion. The cleaning portion has a cleaning member and a heating element. The cleaning member is positioned in the bore such that when the optical instrument is passed through the bore, a lens of the instrument contacts the cleaning member, and the heating element is positioned such that it thermally couples to the lens when the lens is within the bore.

Embodiments of this aspect of the invention can include one or more of the following features. The cleaning member can be a sponge, and the cannula can further include a wetting mechanism, such as a fluid reservoir, arranged to moisten the sponge. The heating element can be a substance that, when triggered, generates an exothermic reaction. The heating element can surround the cleaning member.

In another aspect, the invention features a method of cleaning a lens on an optical surgical instrument. The optical surgical instrument has a distal portion that includes the lens, and the method includes: (a) providing the cannula described above; (b) inserting the distal end of the cannula into a surgical field; (c) passing the distal portion of the optical surgical instrument through the bore of the cannula, into the operating field, and, when the lens becomes covered with fog, fluid, or tissue; (d) withdrawing the distal portion of the instrument from the surgical field into the cleaning portion of the cannula, such that the lens contacts the cleaning member; (e) moving, e.g., rotating, the lens while the lens is in contact with the cleaning member to clean the lens; and (f) reintroducing the distal portion of the instrument into the surgical field.

Different aspects of the invention may include one or more of the following advantages. The self-contained devices are inexpensive to manufacture and sterilize using standard techniques, and can be discarded after a surgical procedure. The devices that use an exothermic chemical heating pad do not require an external power source or a battery, and provide sufficient heat for a sufficient length of time, e.g., for two, four, or six hours or more. The self-contained devices can be pre-assembled. The cannula that includes an integrated cleaning device obviates fully removing the optical instrument from the operating field for cleaning.

As used herein, the term “optical surgical instrument” or “optical instrument” means any instrument used to view an internal portion of a body during a surgical or diagnostic procedure.

Two items that are “thermally coupled” are arranged such that heat can flow from one item to the other. For example, a heating element is thermally coupled to a metal tube if the tube and the heating element are in direct contact, or are in sufficiently close proximity to allow the heating element to heat the tube. The heating element and tube would also be thermally coupled if heat flows from the heating element to the tube through an intermediary heat sink or other heat transfer medium, such as liquid, or heat transfer system.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict of terminology, the present specification, including definitions, will control. In addition, the materials, methods, and examples are illustrative only and are not intended to be limiting.

Other features and advantages of the invention will be apparent from the following detailed description, and from the claims.

### BRIEF DESCRIPTION OF THE DRAWINGS

FIG. 1A is a perspective view of a laparoscope, with an associated camera and video coupler.

FIG. 1B is an enlarged, perspective view of a distal portion of the laparoscope of FIG. 1A.

FIG. 2A is an exploded view of a lens warming and cleaning device.

FIGS. 2B and 2C are perspective views of the device of FIG. 2A.

FIG. 2D is a perspective view of the device of FIG. 2A with a spring clip attached.

FIG. 2E is a perspective view of a drapery grabber for use with the device of FIG. 2A.

FIG. 2F is a perspective view of the device of FIG. 2A with a hook and loop fastener assembly for attaching the device to a surgical table.

FIG. 3A is an exploded view of a second lens warming and cleaning device.

FIG. 3B is a perspective view of the device of FIG. 3A.

FIG. 4A is a sectional view of a third lens warming and cleaning device.

FIG. 4B is a perspective view of the device of FIG. 4A, showing the device's hinged top open, and illustrating use of the device.

FIG. 4C is a perspective view of the device of FIG. 4A, showing the device's hinged top closed.

FIG. 5A is an exploded view of a fourth lens warming and cleaning device.

FIG. 5B is a perspective view of the device of FIG. 5A, illustrating assembly of the device.

FIGS. 5C and 5D are perspective views of the device of FIG. 5A, showing the device fully assembled.

FIG. 5E is a perspective view of a shell for containing the device of FIG. 5A.

FIG. 5F is an exploded view of the device of FIG. 5A and the shell of FIG. 5E, showing insertion of the device into the shell.

FIG. 5G is a perspective view of the device of FIG. 5A fully inserted into the shell of FIG. 5E.

FIG. 6A is a perspective view of a trocar that includes an integrated lens warming and cleaning compartment.

FIG. 6B is a sectional view of the trocar of FIG. 6A, taken along the line B-B of FIG. 6A.

### DETAILED DESCRIPTION

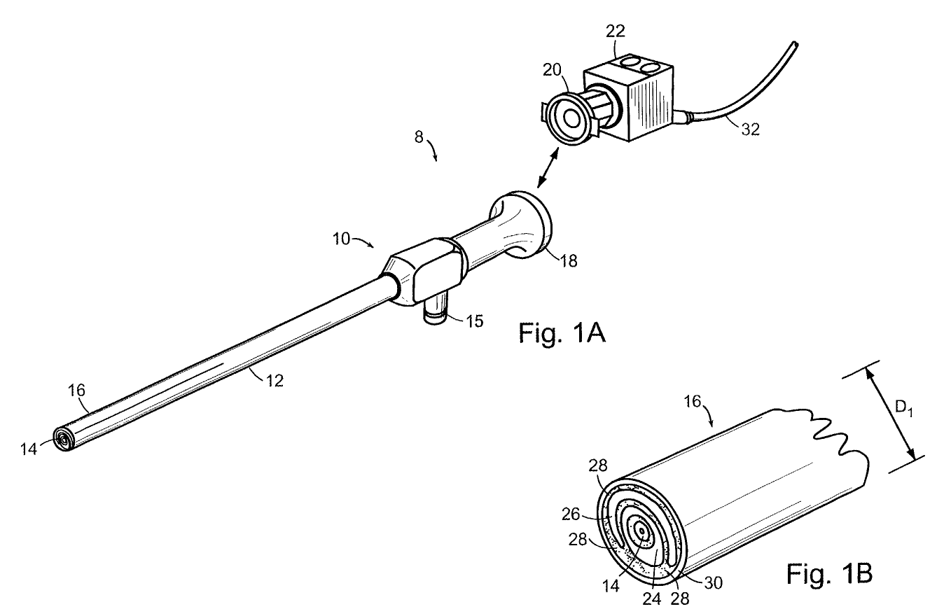

The new warming and cleaning devices are used with standard optical instruments. Referring to FIG. 1A, a typical laparoscope system 8 includes a laparoscope 10, a video coupler 20, a camera 22, and a video display (not shown). Laparoscope 10 includes a shaft 12, a lens 14, a light source input coupler 15, and a proximal end 18 for coupling to the camera.

FIG. 1B provides an enlarged view of a distal portion 16 of shaft 12, and illustrates the positioning of lens 14 within the laparoscope. As shown in FIG. 1B, lens 14 is located in the center of shaft 12, and is surrounded by an end plate 24 and a fiber optic ring 26. An epoxy potting material 28 separates end plate 24 from fiber optic ring 26, and separates the fiber optic ring from the stainless steel shell 30 of shaft 12. The potting material 28 tends to thermally isolate lens 14 from shell 30 of shaft 12. Distal portion 16 has an overall diameter D₁.

In operation, video coupler 20 is attached to proximal end 18 of laparoscope 10, allowing camera 22 to receive images from lens 14. A cable 32 connects camera 22 to the video display (not shown). During surgery, laparoscope 10 is inserted, distal portion 16 first, into an intracorporeal operating field, such as an insufflated abdomen. Laparoscope 10 delivers images of the operating field captured by lens 14 to camera 22, and ultimately to the video display, allowing a surgeon to visualize the field. Endoscopes and other optical instruments have similar features.

The new devices for warming and cleaning lens 14 of laparoscope 10 (or similar optical instruments) can be compact, self-contained units, or can be integrated into a trocar-cannula system. In either case, they can be manufactured using standard medical device manufacturing technologies. The devices can be manufactured under sterile conditions, or can be sterilized after manufacture and before or after packaging using standard techniques.

Self-Contained Warming and Cleaning Devices

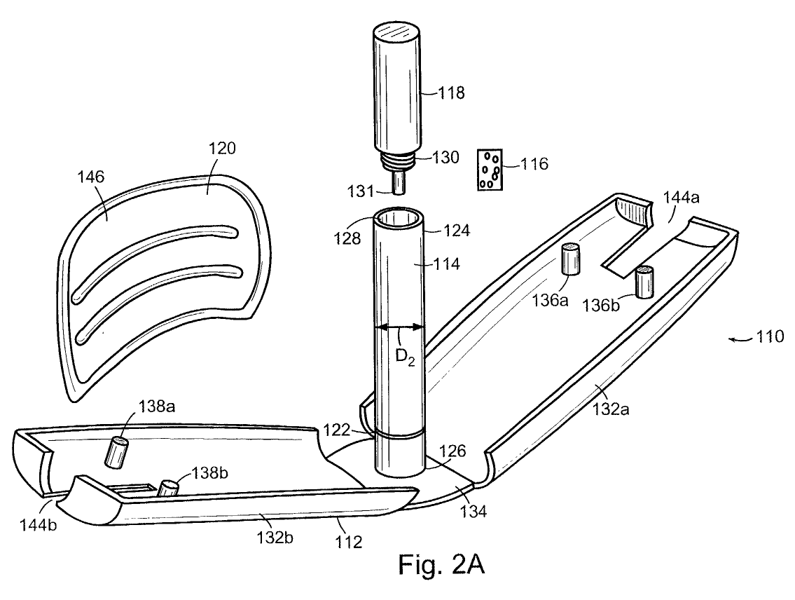

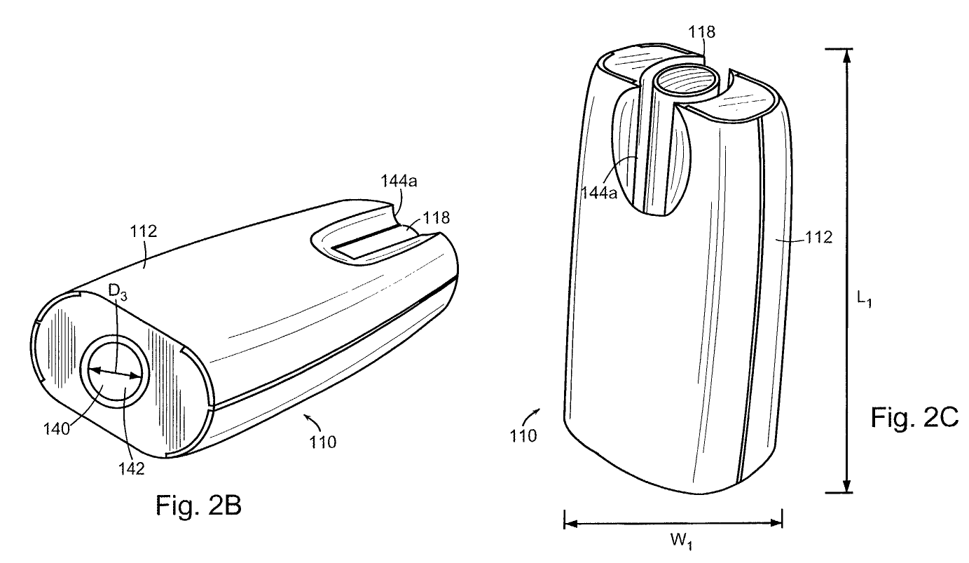

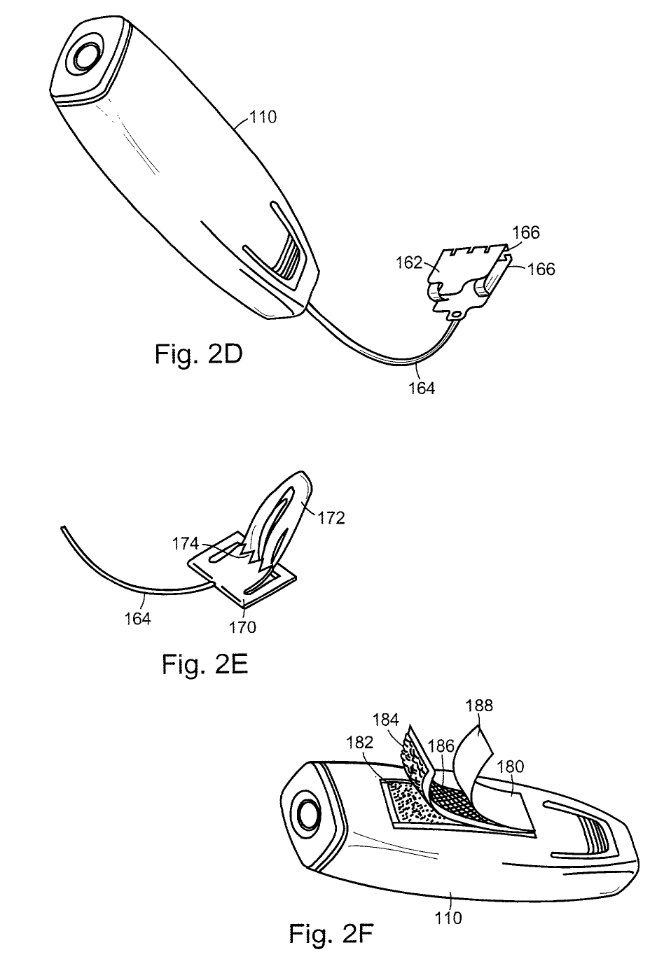

FIGS. 2A to 5F illustrate four different embodiments of the new compact, self-contained devices for warming and cleaning lens 14 of laparoscope 10 or other optical instruments.

Referring to FIGS. 2A to 2C, a device 110 includes a housing 112, a heat-conducting tube 114, a sponge 116, a squeezable container or bottle 118, and a heating element such as a heating pad 120. Tube 114 has an open proximal end 122, an open distal end 124, and an internal diameter D₂. Diameter D₂ is slightly larger than diameter D₁ of distal portion 16 of laparoscope 10, so that tube 114 can receive distal portion 16.

Distal end 124 is attached to bottle 118, and proximal end 122 is attached to a stem 126 on housing 112. Distal end attaches to bottle 118 via complementary screw threadings 128 (inside tube 114) and 130 (on bottle 118). Alternatively, bottle 118 and distal end 124 can be attached by an interference or press fit, using, e.g., an O-ring. Proximal end 122 is similarly attached to stem 126 using, e.g., complementary screw threadings, an insert mold, or an interference fit.

Sponge 116 is disposed within tube 114, near distal end 124. Sponge 116 is held snugly within tube 114 by, e.g., glue, grooves, or intrusions within tube 114, or an interference or press fit. A dispensing tip 131 of bottle 118 rests against sponge 116.

Housing 112 includes opposing sides 132a, 132b, a base 134, and stem 126. Opposing sides 132a, 132b are held together by, e.g., interference fits between plugs 136a, 136b and plug receivers 138a, 138b, respectively. Base 134 defines a circular opening 140 that has a diameter D₃ approximately equal to or slightly larger than internal diameter D₂ of tube 114. Opening 140 leads to the interior of tube 114 via a bore 142 in stem 126. Bore 142, like opening 140 and tube 114, has a diameter slightly larger than diameter D₁ of distal portion 16.

Opposing sides 132a, 132b of housing 112 define slits 144a, 144b respectively. Slits 144a, 144b allow a surgeon to squeeze bottle 118. Bottle 118 is filled with a cleaning liquid, e.g., water or a biocompatible saline solution. The solution can also contain an anti-fogging element or a surfactant. When the surgeon squeezes bottle 118, the bottle dispenses the saline solution to, moisten sponge 116.

Heating pad 120 in this embodiment includes a flexible, air-permeable outer bag 146 that encases a chemical mixture. The chemical mixture, when activated, generates an exothermic reaction. The chemical mixture can be, e.g., a mixture of iron powder, water, cellulose, vermiculite, activated carbon, and salt. Exposing the mixture to atmospheric oxygen triggers an exothermic reaction that warms pad 120 to a temperature of about 60° C., and sustains that temperature for about six hours.

Other types of known exothermic reaction mixtures can be used. For example, the mixture can consist of iron powder, a chloride or sulfate of a metal having a tendency of ionization greater than iron, active carbon, and water. See, e., Yamashita et al., U.S. Pat. No. 3,976,049. Alternatively, the chemical mixture can be a super-cooled, supersaturated aqueous solution of sodium acetate. See, e.g., Stanley et al., U.S. Pat. No. 4,077,390. Pad 120 can also employ other types of exothermic chemical reactions to generate heat, or it can include a resistance heater powered by, e.g., a battery or an external source of electricity.

Pad 120 is wrapped around tube 114 inside of housing 112. Pad 120 can be attached to tube 114 using any standard fastening methods, e.g., glue. Alternatively, pad 120 can be secured to tube 114 by attaching opposite ends 148a, 148b of pad 120 together using, e.g., an adhesive or hook and loop fasteners, after wrapping pad 120 around tube 114.

Device 110 is assembled by first attaching tube 114 to stem 126. Sponge 116 is then inserted into distal end 124 of tube 114, and bottle 118 is attached to distal end 114 by engaging screw threads 130 with threads 128. Heating pad 120 is removed from a protective seal, and wrapped around tube 114. Pad 120 is secured to tube 114 using, e.g., an adhesive, hook and loop fasteners, sleeves, or a rubber band. Sides 132a, 132b of housing 112 are then snapped together by engaging plugs 136a, 136b with plug receivers 138a, 138b. The entire device 110 is then quickly sealed in a cellophane wrapper (or other suitable, air tight container) to prevent oxygen from reaching pad 120 and continuing the exothermic reaction. Device 110 is generally pre-assembled and pre-sealed at manufacture. To prevent any oxygen from reaching pad 120 during assembly, device 110 can be assembled in, e.g., a nitrogen environment. However, so long as device 110 is sealed in cellophane shortly after pad 120 is removed from its own protective wrapper, a pure nitrogen environment is not necessary.

Housing 112 can be made from, e.g., a hard, inexpensive plastic such as polyethylene or polypropylene. Tube 114 is made from, e.g., aluminum, or some other heat-conducting material. Sponge 116 can be, e.g., porous polyethylene, urea formaldehyde, or other suitable porous material. Alternatively, sponge 116 can be replaced by other types of cleaning members, including other absorbent materials, such as cotton, or a jet spray system. Squeezable bottle 118 can be a suitable plastic, off-the-shelf liquid dispenser.

Device 110 has an overall length L₁ of, e.g., about 5.0-5.5 inches, and an overall width W₁ of, e.g., about 1.0-1.75 inches. Diameters D₁, D₂, and D₃, are, e.g., about 0.4 inches, 0.5 inches, and 0.5 inches respectively. The dimensions of different components in device 110 can be altered to accommodate different types of optical surgical instruments. For example, opening 140, bore 142, and tube 114 need not be cylindrical. The dimensions can also be adjusted to accommodate optical surgical instruments other than those used in minimally invasive procedures.

Device 110 can be used to warm and clean laparoscope 10 in the following manner. First, at the beginning of the surgical procedure, a surgeon or an assistant removes device 110 from its cellophane wrapper, allowing oxygen to reach pad 120 and trigger the exothermic reaction. Sponge 116 is moistened by squeezing bottle 118 to dispense liquid through tip 131 to sponge 116. Laparoscope 10 is then pre-warmed by inserting distal portion 16 of the scope into device 110 through hole 140 and bore 142, until lens 14 abuts sponge 116. Laparoscope 10 is left inside device 110 until the surgeon is ready to insert the scope into the patient.

During a surgical or diagnostic procedure, when lens 14 of laparoscope 10 becomes fogged or covered with fluid or tissue, the surgeon removes the laparoscope from the patient and inserts distal portion 16 of laparoscope 10 through hole 140 and bore 142, until lens 14 abuts sponge 116. The surgeon then gently rubs lens 14 against sponge 116, e.g., by rotating laparoscope 10 about its longitudinal axis, to clean and warm lens 14. Contacting lens 14 against sponge 116 for about 5-60 seconds, e.g., 15-30 seconds will warm lens 14 to a temperature of, e.g., about 45-60° C., a temperature warm enough to prevent condensation, but not so hot that tissue is damaged.

After cleaning, the surgeon withdraws laparoscope 10 from device 110 and reintroduces laparoscope 10 into the intracorporeal operating field. The process can be repeated each time lens 14 becomes fogged or dirty. If necessary, sponge 116 can be re-wetted by again squeezing bottle 118.

Referring to FIGS. 2D to 2F, to simplify access to device 110 during surgery, the device can be attached to a surgical drape, a tray, or a table using, e.g., a clip or hook and loop fasteners. Referring first to FIG. 2D, a spring clip 162 is attached to device 110 by a tether 164. The clip 162 includes teeth 166 for gripping surgical drapery. Referring to FIG. 2E, rather than a clip, a drape grabber 170 can be attached to the device. In use, a portion of the drapery 172 is drawn through grabber 170, so that the grabber's teeth 174 hold the drapery fold 172 in place. Referring to FIG. 2F, a hook and loop assembly 180 can be used to attach the device to an operating table. The assembly 180 includes a hook sheet 182 attached to device 110, and a loop sheet 184. Loop sheet 184 has an adhesive backing 186 for attaching the loop sheet to an operating table. A cover 188 covers adhesive backing 186 prior to use. In use, cover 188 is removed from loop sheet 184, and adhesive backing 186 is attached to the table. During surgery, device 110 can be attached to the table by coupling hook sheet 182 to loop sheet 184.

Device 110 can also be modified in other ways. For example, moistening mechanisms other than a squeezable bottle, e.g., a fluid reservoir built into the device can be used to wet sponge 116. Heating pad 120 can be replaced by other types of heating elements, including resistance powered heaters thermally coupled to heat conducting tube 114.

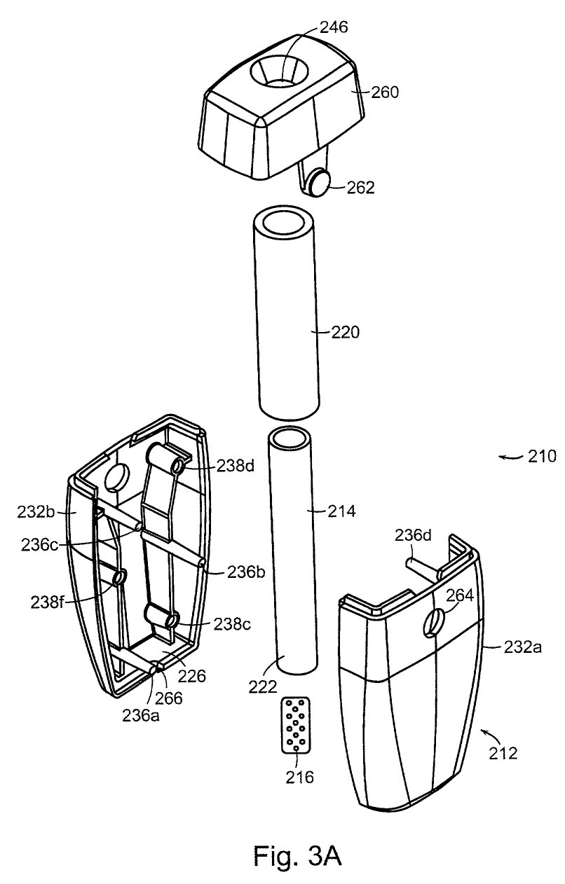

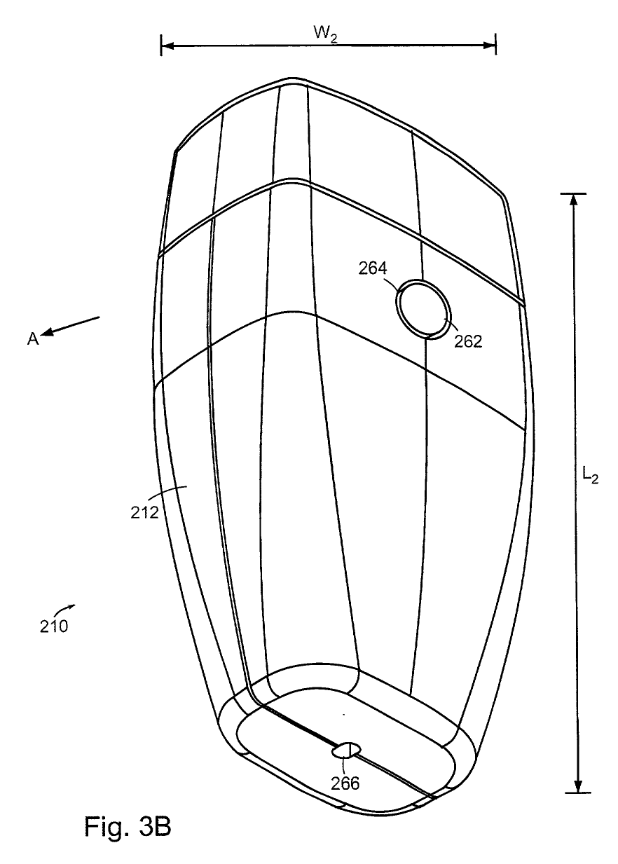

FIGS. 3A and 3B illustrate a second lens warming and cleaning device that has a three piece housing with an easily removable top, and no integrated saline bottle. Referring to FIGS. 3A and 3B, device 210 includes a housing 212, a heat-conducting tube 214, a sponge 216, and a heating pad 220. Tube 214, sponge 216, and pad 220 are similar to tube 114, sponge 116, and pad 120 described above. Housing 212 has three components: sides 232a and 232b, and top 260. Sides 232a, 232b attach together by engaging plugs 236a, 236b, 236c, 236d, and two additional plugs (not shown), with plug receivers 238d, 238e, 238f, and three additional plug receivers (not shown). Unlike housing 112, housing 212 does not include a stem 126 for engaging a proximal end 222 of tube 214. Instead, proximal end 222 rests against a floor 226 of sides 232a and 232b.

Top 260 fits over sides 232a and 232b, and attaches to side 232a by engaging button 262 with button hole 264. The engagement of button 262 with button hole 264 is easily reversible by simply pressing button 262 inward, in the direction of arrow A, as shown in FIG. 3B. Top 260, therefore, can be easily removed, allowing replacement of individual components of device 210, such as tube 214, pad 220, or sponge 216. Device 210, therefore, need not be entirely discarded when pad 220 loses its heat, or sponge 216 becomes inaccessible or dirty.

Device 210 does not include an integral solution bottle. Instead, floor 226 defines a liquid hole 266 in communication with sponge 216. A user, therefore, can wet sponge 216 by squirting, pouring, or dripping liquid through hole 266. Alternatively, device 210 can have a solid bottom (no hole 266), and sponge 216 can be wetted by pouring liquid through hole 240.

In operation, distal portion 16 of laparoscope 10 is inserted through opening 240 until lens portion 14 of the laparoscope abuts sponge 216. The lens is then cleaned and warmed in the manner described above with reference to device 110.

Device 210 has an overall length L₂ slightly shorter than length L₁ of device 110, since device 210 lacks a solution bottle 118. The width W₂ of device 210 is approximately equal to width W₁ of device 110.

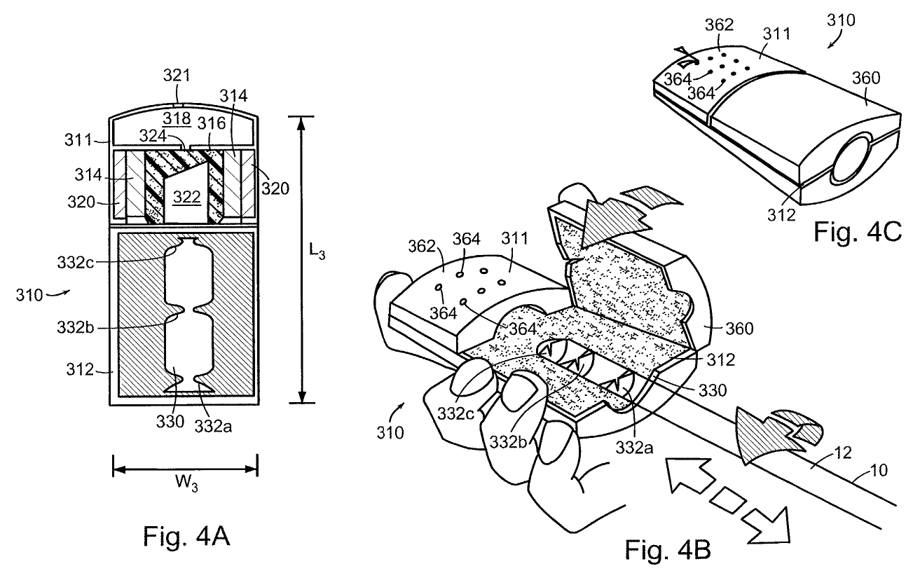

FIGS. 4A to 4C illustrate a third embodiment of the lens warming and cleaning device that includes separate wiping and cleaning compartments. Device 310 has a cleaning compartment 311 and a wiping compartment 312. Cleaning compartment 311 includes a heating pad 320, a sponge 316, and a fluid reservoir 318. A corked or otherwise re-sealable hole 321 allows access to reservoir 318, so that fluid in the reservoir can be replenished.

Sponge 316 has a generally cylindrical shape, defining a bore 322 for receiving distal portion 16 of laparoscope 10. A port 324 connects fluid reservoir 318 to sponge 316, so that fluid from reservoir 318 keeps sponge 316 moist. Pressing distal portion 16 of scope 10 against sponge 316 draws fluid from reservoir 318 to sponge 316, by capillary action. Heating pad 320 and sponge 316 are separated by a heat-conducting tube 314.

Wiping compartment 312 defines a wiping groove 330. Groove 330 includes rubber wipers 332a, 332b, and 332c for wiping fluid and tissue from shaft 12 of laparoscope 10, as shown in FIG. 4B. Wiping compartment 312 includes a hinged top 360 that opens, providing access to wiping compartment 312. Opening hinged top 360 allows a user to gain access to the interior of compartment 312, e.g., to clean wipers 332a, 332b, and 332c.

A cover 362 for cleaning compartment 311 defines apertures 364, which allow oxygen to reach heating pad 320, triggering the exothermic reaction described above with reference to pad 120.

Device 310 has an overall length L₃ slightly longer than devices 110 and 210. Length L₃ is, e.g., about seven inches. Device 310 has an overall width W₃ similar to widths W₁ and W₂ of devices 100 and 210.

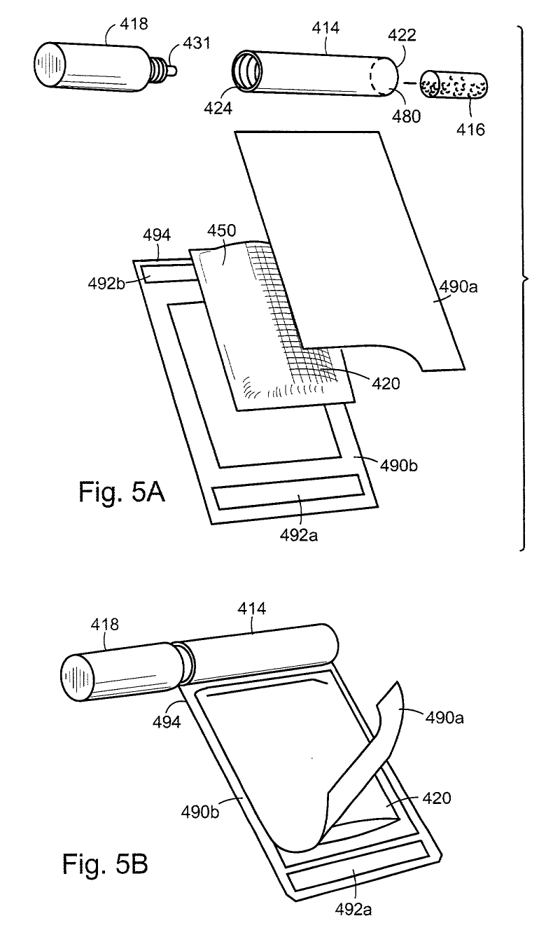

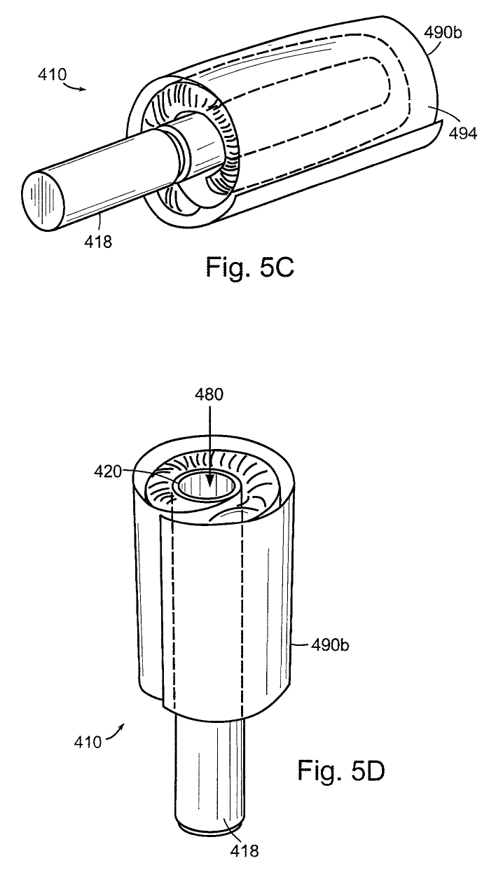

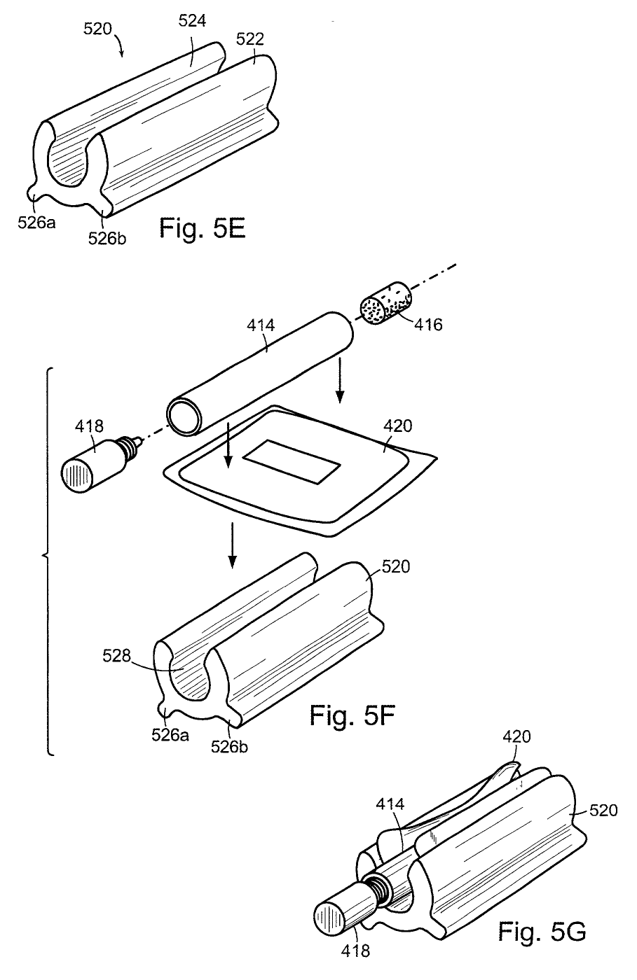

FIGS. 5A-5G illustrate a simplified, inexpensive warming and cleaning device 410 that lacks an enclosed external housing. Referring first to FIGS. 5A-5D, device 410 includes a heat-conducting tube 414, a sponge 416, a squeezable saline bottle 418, and heating pad 420. Like tube 114 of FIGS. 2A-2C, tube 414 includes an open proximal end 422 and an open distal end 424. Opening 480 of open proximal end 422 is sized and shaped to receive distal portion 16 of laparoscope 10, and open distal end 424 is configured to attach to bottle 41 8. Sponge 416 is disposed within tube 414, near distal end 424, touching a dispensing tip 431 of bottle 418.

Unlike devices 110, 210, and 310, the heating pad of device 410 is generally not attached to the device at the time of manufacture. Instead, the user attaches heating pad 420 immediately prior to use. Referring to FIGS. 5A and 5B, prior to assembly by a surgeon or an assistant, pad 420 is enclosed by two seals, 490a and 490b. Seals 490a, 490b are attached to each other by double-sided adhesive strips 492a, 492b.

Just before surgery, the surgeon or an assistant removes seal 490a, exposing air-permeable cover 450 of pad 420 to the air. Oxygen penetrates cover 450, triggering the exothermic reaction described above with reference to pad 120.

After removing seal 490a, the surgeon or assistant rolls pad 420 and seal 490b around tube 414, as shown in FIG. 5B. As pad 420 and seal 490b are rolled around tube 414, adhesive 492b attaches to tube 414, and adhesive 492a, attaches to a far end 494 of seal 490b, trapping pad 420 in place against tube 414.

The fully rolled device of FIGS. 5C and 5D is ready for immediate use. However, in some situations, it may be preferable to insert the rolled device 410 into a shell to stabilize pad 420 against tube 414 and shield the pad from the exterior. FIGS. 5E to 5G illustrate use of such a shell. Referring to FIG. 5E, a shell 520 has a generally cylindrical body 522, an open top 524 and two wings 526a, 526b. Wings 526a, 526b prevent device 410 from rolling when the device is placed on a flat surface, such as an operating table. Shell 520 is made from a flexible material, e.g., polypropylene, allowing open top 524 to be expanded by pulling wings 526a, 526b apart.

Referring to FIG. 5F, the fully rolled device 410 is inserted into shell 520 by pulling wings 526a, 526b apart, and inserting device 410 into shell 520's cylindrical interior. Alternatively, Shell 520 can be made from a rigid plastic. In a rigid embodiment, rolled device 410 is inserted by sliding device 410 into an open end 528 of shell 520. FIG. 5G shows device 410 fully inserted into shell 520.

Seals 490a, 490b can be made from, e.g., foil, cellophane, or from thin sheets of plastic such as polyethylene or polypropylene, or polyvinylchloride. The remaining components of device 410 can be made from the same materials described above with reference to device 110.

During surgery, device 410 is used in essentially the same manner as device 110. When lens 14 of laparoscope 10 fogs or becomes dirty, the surgeon inserts distal portion 16 through opening 480, into tube 414, until lens 14 abuts sponge 416. After cleaning and warming lens 14 against sponge 416, the surgeon withdraws laparoscope 10 and reintroduces it into the intracorporeal operating field.

Warming and Cleaning Device Integrated into a Cannula

Trocar-cannula systems create channels for introducing instruments, such as laparoscope's or endoscopes, into an intracorporeal operating field. A trocar-cannula system includes a trocar, which is a sharp, pointed surgical instrument that punctures the body, and a cannula. The cannula has a distal end that enters the operating field through the hole formed by the trocar, and a proximal end that remains outside the body. The cannula defines a bore or channel that extends from an opening at the distal end to an opening at the proximal end. Other instruments, such as a laparoscope, are introduced into the surgical field through the channel in the cannula.

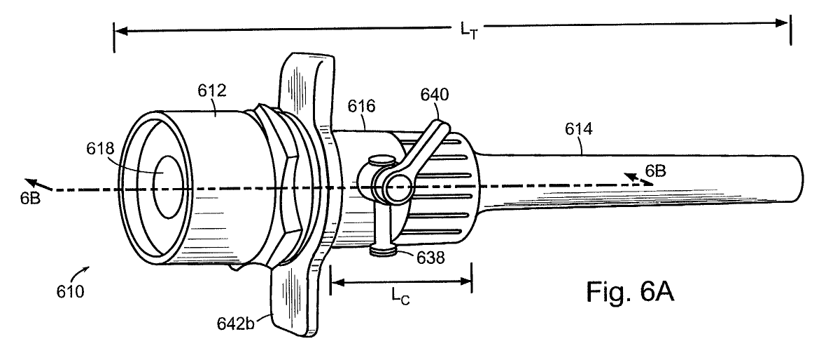

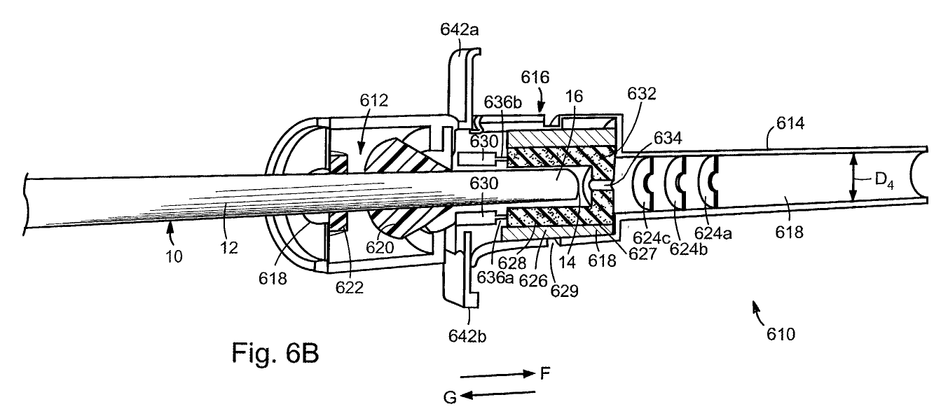

Referring to FIGS. 6A and 6B, a cannula 610 includes a proximal region 612, a distal shaft 614, and a cleaning compartment 616. Proximal region 612, cleaning compartment 616, and distal shaft 614 all define a continuous longitudinal channel 618 for passing surgical instruments, e.g., laparoscope 10, into an intracorporeal operating field.

Proximal region 612 includes a distal seal 620 and a proximal seal 622. Distal seal 620 has a generally conical shape, and is oriented to remove fluid from shaft 12 of laparoscope 10 as laparoscope 10 is moved in the direction of arrow G. Proximal seal 622 has a generally circular shape, and acts to remove fluid and debris from shaft 12 as it is moved in the direction of either arrow F or arrow G. Proximal seal 622 can be, e.g., an O-ring. Seals 620 and 622 can be made from, e.g., silicone or various thermal-plastic rubbers.

Distal shaft 614 includes three interior wipers 624a, 624b, and 624c. Wipers 624a, 624b, and 624c wipe fluid and tissue from distal tip 14 as laparoscope 10 is pulled in the direction of arrow G.

Cleaning compartment 616 includes a heating pad 626, a sponge 628, and a fluid reservoir 630. Sponge 628 has a generally cylindrical shape, surrounding distal portion 16 when the distal portion is inside compartment 616. Sponge 628 includes a circular wall 632. Wall 632 contacts lens 14 as distal portion 16 is moved in the direction of arrow F, out of cleaning compartment 616. A slit 634 in wall 632 allows passage of shaft 12 into and out of cleaning compartment 616.

Fluid reservoir 630 contains, e.g., a saline solution, and acts to keep sponge 628 moist. Ports 636a, 636b allow movement of fluid from reservoir 630 to sponge 628.

Heating pad 626 is similar to the heating pads for the self-contained devices described above. Pad 626 surrounds sponge 628, directly contacting an exterior of sponge 628. To delay activation of pad 626 until surgery, pad 626 is sealed in a permeable membrane 627. Immediately prior to surgery, membrane 627 is punctured by inserting a sharp instrument through access hole 629, thereby exposing pad 626 to oxygen. Alternatively, instead of including a membrane 627, the entire cannula 610 can be sealed in a cellophane wrapper prior to surgery, in the same manner device 110 is sealed.

Cannula 610 also includes an insufflation port 638 and stop cock 640. Insufflation port 638 allows an inert gas to be pumped into an operating field, e.g., an abdominal cavity, to expand the field, making room for surgical instruments. Wings 642a, 642b on cannula 610 limit cannula 610's penetration into the patient.

Cannula 610 has an overall length L_{T} of about 6-12, e.g., 7, 8, 9, or 10 inches, and cleaning compartment 616 has a length L_{C} of about 2-5 inches, e.g., 2, 3, 4, or 5 inches. Channel 618 has an internal diameter D₄ slightly larger than diameter D₁ of shaft 12 of laparoscope 10, e.g., 0.5-1.0 inches.

In use, membrane 627 of heating pad 626 is punctured, and distal shaft 614 is inserted into an intracorporeal operating space. If necessary, the operating field can be expanded using insufflation port 638. Laparoscope 10 is then inserted into the operating field through bore 618. When lens 14 of laparoscope 10 becomes fogged or dirty, distal portion 16 is withdrawn from the patient, in the direction of arrow G, until distal portion 16 is within cleaning compartment 616, as shown in FIG. 6B. Moving distal portion 16 past wipers 624a, 624b, and 624b removes most fluid and tissue from portion 16. Lens 14 is then cleaned and warmed by rubbing lens 14 against wall 632 of sponge 628. Once clean, lens 14 is reintroduced into the patient through distal shaft 614.

A number of features of cannula 610 can be varied. For example, a heat-conducting tube can be inserted between sponge 628 and pad 626, in the manner described above with reference to the self-contained devices. Cleaning compartment 616 can be reduced in size, or can be disposed entirely within shaft 614, thus reducing the length of the cannula protruding out of the body during use. Alternative mechanisms for wetting sponge 628 can be used, including an external squeezable bottle connected to sponge 628 through an opening in the cannula.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A lens warming and cleaning device for use with an optical instrument having a lens portion, the device comprising:
a heat-conducting tube sized and shaped to receive the lens portion of the optical instrument;
a heating element thermally coupled to the tube; and
a cleaning member disposed within the tube, such that when the lens portion of the optical instrument is inserted into the tube, the lens portion contacts the cleaning member.

2. The device of claim 1, wherein the heat conducting tube is sized and shaped to receive the lens portion of an optical instrument selected from the group consisting of a laparoscope and an endoscope.

3. The device of claim 1, wherein the tube is constructed from a material comprising aluminum.

4. The device of claim 1, wherein the heating element comprises a substance that, when triggered, generates an exothermic reaction.

5. The device of claim 4, wherein the substance comprises a mixture of compounds, and the exothermic reaction is triggered by exposing the substance to oxygen.

6. The device of claim 1, wherein the heating element comprises a flexible pad.

7. The device of claim 6, wherein the pad surrounds at least a portion of the tube that receives the lens portion of the optical instrument.

8. The device of claim 7, wherein the pad comprises an attachment mechanism, the attachment mechanism securing the pad to the tube.

9. The device of claim 8, wherein the attachment mechanism comprises an adhesive.

10. The device of claim 1, wherein the heating element generates sufficient heat to warm the lens portion of the optical instrument to between about 45° C. and 60° C.

11. The device of claim 1, wherein the cleaning member comprises a sponge.

12. The device of claim 11, wherein the sponge is disposed within a distal end of the tube.

13. The device of claim 11, further comprising a moistening mechanism that moistens the sponge.

14. The device of claim 13, wherein the moistening mechanism comprises a squeezable bottle containing a liquid.

15. The device of claim 14, wherein the liquid is a saline solution

16. The device of claim 15, wherein the saline solution comprises an anti-fogging agent, a surfactant, or both.

17. The device of claim 1, further comprising a housing that encases the tube and the heating element.

18. The device of claim 17, wherein the housing defines an opening configured for insertion of the lens portion of the optical instrument.

19. The device of claim 17, further comprising an attachment mechanism coupled to the housing, the attachment mechanism having a member configured to removably attach the device to a table or to a surgical drape.

20. The device of claim 19, wherein the member comprises a clip.

21. The device of claim 20, wherein the attachment mechanism further comprises a tether that connects the clip to the housing.

22. The device of claim 19, wherein the attachment mechanism comprises a hook and loop fastener system, the system having a first sheet attached to the housing, and a second sheet attached to a table or to a surgical drape, the first sheet comprising one of hooks or loops, and a second sheet comprising the other of hooks or loops.

23. A lens warming and cleaning device for use with an optical instrument having a lens portion, the device comprising:
a heat-conducting tube sized and shaped to receive the lens portion of the optical instrument;
a flexible heating pad wrapped around at least a portion of the tube, the heating pad comprising a substance that, when triggered, generates an exothermic reaction; and
a sponge disposed within a distal end of the tube, such that when the lens portion of the optical instrument is inserted into the tube, the lens portion contacts the sponge.

24. The device of claim 23, further comprising a wetting mechanism arranged to moisten the sponge.

25. A method of warming and cleaning a lens of an optical instrument, the method comprising:
obtaining the device of claim 1;
withdrawing the optical instrument from an operating field;
inserting a lens portion of the optical instrument into the tube such that the lens contacts the cleaning member; and
moving the lens while the lens is in contact with the cleaning member to remove fog and debris from a surface of the lens.

26. The method of claim 25, wherein the inserting step includes inserting only a distal portion of the optical instrument into the tube, the distal portion comprising the lens.

27. The method of claim 25, further comprising:
removing the optical instrument from the tube; and
reintroducing the optical instrument into the operating field, and then repeating the withdrawing, inserting, and removing steps when the lens portion again requires cleaning.

28. A cannula comprising:
a body having a distal end and a proximal end, the body defining a bore for passage of an optical instrument therethrough; and
a cleaning portion disposed within the bore between the distal end and the proximal end, the cleaning portion comprising a cleaning member and a heating element, wherein the cleaning member is positioned in the bore such that when the optical instrument is passed through the bore, a lens of the instrument contacts the cleaning member, and wherein the heating element is positioned such that it thermally couples to the lens when the lens is within the bore.

29. The cannula of claim 28, wherein the cleaning member comprises a sponge.

30. The cannula of claim 28, further comprising a wetting mechanism arranged to moisten the sponge.

31. The cannula of claim 30, wherein the wetting mechanism comprises a fluid reservoir in fluid communication with the sponge.

32. The cannula of claim 28, wherein the heating element comprises a substance that, when triggered, generates an exothermic reaction.

33. The cannula of claim 28, wherein the heating element surrounds the cleaning member.

34. A method of cleaning a lens on an optical surgical instrument, the optical surgical instrument having a distal portion that includes the lens, and the method comprising:
providing the cannula of claim 28;
inserting the distal end of the cannula into the operating field;
passing the distal portion of the optical surgical instrument through the bore of the cannula, into the operating field, and, when the lens becomes covered with fog, fluid, or tissue;
withdrawing the distal portion of the instrument from the surgical field into the cleaning portion of the cannula, such that the lens contacts the cleaning member;
moving the lens while the lens is in contact with the cleaning member to clean the lens; and
reintroducing the distal portion of the instrument into the surgical field.

35. The method of claim 34, wherein the cleaning member comprises a sponge.
